(19) United States
(12) Patent Application Publication
(10) Pub. No.: US 2007/0239037 A1
(43) Pub. Date: Oct. 11, 2007

(54) INTERVENTRICULAR DELAY AS A PROGNOSTIC MARKER FOR REVERSE REMODELING OUTCOME FROM CARDIAC RESYNCHRONIZATION THERAPY

(76) Inventors: Stefano Ghio, Milano (IT); Nick Freemantle, Worcestershire (GB); John G. F. Cleland, Kingston Upon Hull (GB); Luigi Tavazzi, Pavia (IT)

Correspondence Address: MEDTRONIC, INC., 710 MEDTRONIC PARKWAY NE, MINNEAPOLIS, MN, 55432-9924, US

(21) Appl. No.: 11/538,210

(22) Filed: Oct. 3, 2006

Related U.S. Application Data
(60) Provisional application No. 60/723,785, filed on Oct. 5, 2005.

(51) Int. Cl. A61N 1/05

(52) U.S. Cl. 600/481; 607/9

(57) ABSTRACT

In heart failure (HF) patients diagnosed according to the New York Heart Association (NYHA) rating scale as either Class III or Class IV, with QRS duration ≧120 ms, left ventricular (LV) EF≦35% (when in sinus rhythm), cardiac resynchronization therapy (CRT) can predict sustained improvement in at least LV structure and function. The knowledge of aetiology of a subject's HF status and of a few simple echocardiographic characteristics provides useful information as to whether such patients and undergo significant and beneficial reverse remodelling of the LV in particular and, in the long term can be expected to experience an improvement in all-cause mortality, for example such patients can be reasonably expected to survive and enjoy a relatively enhanced quality of life (QOL). A patient who qualifies according to the invention can be termed a reverse remodelling responder (RRR) or the like.

### FIELD OF THE INVENTION

The present invention relates to the field of cardiac resynchronization therapy (CRT); in particular, the invention relates to methods and apparatus for predicting whether a given subject will respond superbly to chronic deliver of CRT.

### BACKGROUND

Reverse remodelling is phrase that has been used to indicate a positive therapeutic response to a form of heart failure therapy; namely, CRT. Although CRT has been alleged to induce reverse remodelling the inventors are not aware of any methods or apparatus for predicting whether a given subject. Herein, the phrase reverse remodelling is intended to refer to a process whereby all or a portion of a human heart is restored to a relatively healthy size, function, chamber dimension, valve and/or contractile performance and the like. Alternatively, reverse remodelling can include, without limitation, instigating a less pathophysiologic condition for all or a portion of the human heart for example, a failing dilated left ventricle (LV).

Intervals of CRT delivery or substantially continuous CRT delivery are both known in the art; for example U.S. Pat. No. 6,842,642 to Vanhout describes and claims the use of ratios of CRT delivery based on a number of sensed cardiac events. In addition, U.S. Pat. No. 6,839,592 to Grandjean describes and claims use of various timing intervals in order to improve a patient's hemodynamic performance during CRT delivery. The '642 and '592 patents are hereby incorporated herein by reference in their respective entireties. Furthermore, a published U.S. patent application to Burnes and Mullen describes a novel form of CRT wherein a single ventricular activation produces a fusion depolarization with an intrinsic wavefront. This application was published as U.S. Patent Application No. 20050209648 on 22 Sep. 2005 and is entitled, “Apparatus and Methods of Energy Efficient, Atrial-based Bi-Ventricular Fusion-pacing,” the contents of which are hereby incorporated herein.

With respect to echocardiography methods and apparatus as contemplated and employed in conjunction with the present invention, U.S. Pat. No. 6,514,207 entitled, “Method and Apparatus for Processing Echocardiogram Video Images,” is incorporated herein.

Also, U.S. Pat. No. 5,370,122 entitled, “Method and Apparatus for Measuring Myocardial Impairment, Dysfunctions, Sufficiency, and Insufficiency,” is incorporated herein by reference in its entirety.

### BRIEF SUMMARY

According to the present invention, a clinician or physician can determine whether a given subject can be reasonably expected to respond to CRT and thus benefit CRT delivery resulting in or temporary reverse remodelling. In one form of the invention, a subject's inter-ventricular mechanical delay (IVMD) includes a substantially linear relationship for heart failure (HF) patients who also suffer from a variety of related and oftentimes aggravating co-morbidities (e.g., ischemia).

Doppler echocardiographic examinations were performed at baseline (e.g., just prior to receiving CRT delivery and/or during implantation of an implantable medical device (IMD) capable of CRT delivery), at three months, at 18 months, and at the end of the study (average duration of 29 months for the subjects) in 735 subjects enrolled in the Cardiac Resynchronization Heart Failure Study (CARE-HF trial) and randomized 1:1 to multi-site pacing and control. Echocardiographic recordings were submitted to a third party to help ensure consistency of quantitative analysis.

The CRT group showed progressive improvement in LV end-diastolic volume, end-systolic volume, and ejection fraction (EF) over the entire follow-up period. The extent of reverse remodelling was greater in non-ischemic than in ischemic patients.

The improvement with CRT was attenuated for subjects having right ventricular (RV) dysfunction but not attenuated for subjects having a restrictive LV filling pattern at baseline. The probability of surviving and showing significant reverse remodelling at 18 months was linearly related to IVMD in HF subjects presenting with and without ischemic heart disease (IHD).

According to the invention, chronic delivery of CRT can be employed to advantageously precipitate long term reverse remodelling of the failing LV or other chamber of a person suffering from HF. Simply utilizing knowledge of the etiology of HF and of a few echocardiographic characteristics provides useful information as to whether such patients will be responders to CRT in the long term.

### EXTENDED SUMMARY OF METHODS, CLINICAL TECHNIQUES, and APPARATUS

In patients with heart failure (HF), all pharmacological agents which reduce mortality and morbidity, such as beta blockers and ACE-inhibitors, also improve geometry and function of the LV (1-6). Although the precise relationship linking changes in heart function to improvement or worsening in prognosis has not been fully clarified, reverse remodelling is regarded as a valuable marker of therapeutic response to a heart failure therapy. The CARE-HF study demonstrated that CRT improves symptoms and quality of life (QOL) and reduces the risk of death in NYHA class III or IV patients with systolic HF, broad QRS, left bundle branch block and in sinus rhythm. All patients enrolled in the CARE-HF trial underwent a Doppler echocardiographic examination at baseline and during a follow-up period which continued for at least 18 months after the last patient had been enrolled. This provides a unique opportunity to assess the long-term effects of CRT on LV (LV) structure and function in a large, controlled population of HF patients.

The goal of this prospective echocardiographic study was therefore to address some of the issues which remain as yet unresolved as regards the effects of CRT on cardiac function. First, whether reverse remodelling is sustained over the long term. Second, whether the magnitude of the benefit obtainable with CRT is lower in patients with ischemic aetiology and whether there are patients “too sick” to benefit from CRT. Finally, we examined the hypothesis that the inter-ventricular mechanical delay (IVMD), a dyssynchrony parameter easily obtainable with a standard echocardiographic examination, may be used to predict the long-term responsiveness to CRT. Such information is of outmost importance to clinicians who have to decide which patients need to be implanted with biventricular devices: in fact, although the CARE-HF study demonstrated no heterogeneity in the beneficial effects of CRT according to several pre-specified characteristics of the patients, refinement of selection criteria is crucial to reduce risks and improve the cost-effectiveness of therapy.

Methods

Study Population

The Cardiac Resynchronization-Heart Failure (CARE-HF) trial was a multi-center, international, randomized trial comparing the effect of cardiac resynchronization in addition to standard pharmacologic therapy versus standard pharmacological therapy in 813 patients with New York Heart Association (NYHA) class III or IV, QRS duration ≧120 ms, LV ejection fraction (EF)≦35%, LV end diastolic diameter ≧30 mm/m (height) and sinus rhythm. Patients were randomised 1:1 to multi-site pacing or medical therapy alone. Of the 813 patients enrolled in the trial, 735 had an analyzable echocardiographic examination at baseline and constitute the population described in the present paper (365 patients randomised to CRT and 370 patients randomised to control group).

Echocardiographic Procedures

The echocardiographic qualification process, the recording and analysis of echocardiographic examinations have been previously described (14). Briefly, to participate in the CARE-HF trial, each centre had to go through a qualification procedure to assess accuracy and reproducibility. Echocardiographic recordings were obtained with the use of commercially available instruments and recorded on Super VHS videotapes at baseline and during follow-up at three month, at 18 month and at end of study. Quantitative analysis was performed in a Core Echo Laboratory to ensure blindness in the analysis and consistent measurement methodology. Each variable was measured three times, and the average value calculated. All readings were made by qualified technicians and subsequently reviewed by a senior echocardiographer.

The end-diastolic (EDV) and end-systolic (ESV) volumes of the LV were measured using the single plane area-length method; ejection fraction (EF) was calculated as follows: (EDV-ESV)/EDV×100%.

The degree of mitral regurgitation (MR) was assessed as the area of the colour Doppler regurgitant jet divided by the area of the left atrium in systole. Pulsed Doppler trans-mitral flow velocity curves were recorded at 100 mm/sec and a deceleration time (DT) of the E wave <115 ms was considered indicative of a restrictive LV filling pattern. The tricuspid annular plane systolic excursion (TAPSE) was used as an indicator of RV function. The inter-ventricular mechanical delay (IVMD), calculated as the time difference between the onset of forward flow in the LV (APET) and in the RV (PPET) outflow tracts, was used as index of inter-ventricular mechanical dyssynchrony.

Statistical Analysis

Relevant echocardiographic variables were described at baseline and each time period by group. Differences in mean values for each time period were estimated using mixed models, accounting for treatment and baseline values as patient level covariates, and investigational sites as random effects. Prespecified interaction terms were investigated. A non linear model was used to investigate the extent to which prespecified patient characteristics predicted response at 18 months. Variables were identified for inclusion in the final model using stepwise selection.

Interactions between CRT effects and the following prospectively identified baseline characteristics were studied, for example, etiology of HF (ischemic versus non ischemic), LV filling pattern (DT less than or equal to or greater than 115 ms), RV dysfunction (tricuspid annular plane systolic excursion (TAPSE) less than or equal to greater than 14 mm).

A long-term “reverse remodelling responder” (RRR) to CRT was defined as a patient who survived and whose ESV was reduced at the 18 months evaluation by at least 40 ml. Only death was included in this response outcome measure because in a long-term follow-up “soft” end-points could be misleading. As far as the echocardiographic criterion is concerned, it was derived from variability data obtained in the echocardiographic core laboratory: Since inter-observer variability in the measurement of ESV in the core laboratory was 8.0±19.9 ml, an RRR was defined as a patient whose ESV was reduced by at least 40 ml (twice the standard deviation). It was decided to use the 18 month evaluations both in the prediction of long-term responders and in the analysis of interactions between CRT effects and clinical/echocardiographic parameters (rather than the end-of-study recordings), since the number of patients having an end-of-study examination was substantially smaller and this reduced the power of statistical analysis.

Select Results

Long term reverse remodelling. FIG. 9 is a table showing the echocardiographic measurements at baseline, three month, 18 month and 29 month (end of study) in the control and CRT groups. FIGS. 2, 3 and 4 show the net benefits in echocardiographic parameters obtained with CRT (i.e. the changes with respect to baseline in CRT patients surviving at each time point in follow-up minus the changes observed in control patients).

Mitral regurgitation decreased in CRT patients; the maximal change was observed at three months (−4.9 units, 95% Cl −7.2 to −2.6, p<0.001) with no further improvement at 18 months (−4.2 units, 95% Cl −7.0 to −1.4, p=0.004) and at 29 months (−4.0 units, −7.2 to −0.8, p=0.015) (FIG. 1). On the contrary, geometry and function of the LV showed a progressive improvement in the CRT group over the entire follow-up period. As compared to the control group, EDV was reduced by 33.4 ml (95% Cl −40 to −26.8, p<.0.0001) at three months, by 46.3 ml (95% Cl −57.8 to −34.9, p<0.0001) at 18 months and by 57.6 ml (95% Cl −71.8 to −43.4, p<0.0001) at 29 months (FIG. 2). ESV was reduced by 33.5 ml (95% Cl −39.2 to −27.8, p<0.0001) at three months, by 47.2 ml (95% Cl −57.3 to −37.2, p<0.0001) at 18 months and by 55.1 ml (95% Cl −67.2 to −42.9, p<0.0001) at 29 months (FIG. 3). EF increased by 3.7 EF units (95% Cl 3.0 to 4.4, p<0.0001) at 3 months, by 6.9 EF units (95% Cl 5.6 to 8.1, p<0.0001) at 18 months and by 7.0 EF units (95% Cl 5.5 to 8.5, p<0.0001) at 29 months (FIG. 4).

According to the definition of long-term responsiveness above specified, the percentage of long term responders was 49.2% at 18 months in the CRT group as compared to 18.6% at 18 months in the control group (p<0.001).

Effects of CRT according to Aetiology

A statistically significant interaction was found between ischemic aetiology and the effects of treatment on several echocardiographic parameters. The effect of CRT on EDV was reduced by 15.8 ml at 3 months (p=0.020) and by 27.5 ml (p=0.021) at 18 months in patients with ischemic aetiology as compared to non ischemic aetiology. The effect of CRT on ESV was reduced by 12.4 ml at 3 months (p=0.035) and by 25.6 ml (p=0.014) at 18 months in patients with ischemic aetiology. In terms of EF improvements, there was no difference at three months; however, at 18 months the effect of CRT was reduced by 3.8 EF units in patients with ischemic aetiology as compared to non ischemic aetiology (p=0.003). No interaction was found between aetiology of HF and the reduction of mitral regurgitation with CRT.

Effects of CRT in Patients with Restrictive Filling Pattern and in Patients with RV Dysfunction

Essentially no interaction was found between LV filling pattern and either the reduction of LV volumes or the improvement in EF obtained at three months and at 18 months with CRT delivery. A statistically significant relationship was found between TAPSE and the effects of treatment at 18 months. In patients with a TAPSE less than 14 mm at baseline the effect of CRT on EDV was reduced by 56.1 ml (p=0.004), the effect on ESV was reduced by 52.4 ml (p=0.002) and the effect on EF was reduced by 6.8 EF units as compared to patients with TAPSE ≧14 mm (p=0.003).

Inter-Ventricular Dyssynchrony and Prediction of Long Term Responsiveness

As compared to the control group, IVMD was reduced by 21.2 ms (95% Cl −24.7 to −17.7, p<0.0001) at three months, by 20.7 ms (95% Cl −24.9 to −16.5, p<0.0001) at 18 months and by 21 ms (95% Cl −26.2 to −15.9, p<0.0001) at 29 months. In a multivariate model, IVMD was significantly related to the outcome at 18 months, as was the aetiology of HF (ischemic versus non ischemic), (FIG. 10). The probabilities of response according to IVMD at baseline, aetiology of heart failure and treatment are shown in FIG. 5. The probability of being a responder increased linearly with the increase in IVMD, regardless of aetiology. In absolute terms, IHD patients were less likely to be considered responders.

### BRIEF DISCUSSION

The results of the present study demonstrate that CRT determines a sustained, long term improvement of LV structure and function of the failing LV. The extent of reverse remodelling is lower in patients with IHD and in patients with severe RV dysfunction. A higher inter-ventricular mechanical delay at baseline suggests a higher probability of surviving and having significant remodelling with long-term CRT.

Long Term Reverse Remodelling

The CARE-HF study demonstrated that CRT improves symptoms and quality of life (QOL) and reduces the risk of death in NYHA Class III or Class IV patients with systolic HF, broad QRS, left bundle branch block and in sinus rhythm. The results of the present echocardiographic study that were used in the development of the present invention indicate that sustained reverse remodelling of the failing LV is the plausible biological explanation for such an improvement in morbidity and mortality. The evidence for LV reverse remodelling was in fact physiologically sound, since the reduction in EDV and ESV was associated with an improvement in both EF and in mitral regurgitation, supporting the concept of an increase in cardiac output. The extent of LV reverse remodelling was not subtle; despite the fact that CRT was added to optional medical therapy the increase in EF and the decrease in EDV and ESV with CRT were similar or even slightly higher then those reported in trials with medical therapy (e.g., administration of beta-blockers) in HF patients. For RRR patients reverse remodelling was sustained over the entire follow-up period; importantly, the 29 months average follow-up in the present study is the longest follow-up reported in trials aimed at evaluating the effects of CRT on LV structure and function. A slight reduction in LV volumes and an increase in EF was observed also in the control group; however, this result is likely due to the fact that some of the most compromised patients succumbed during the long term follow-up and the surviving subjects had better overall cardiac function. Thus, the inventors conclude that with careful monitoring, medical care and optimal compliance with an appropriate therapeutic regimen (whether pharmacological treatment or similar) can induce good results even in HF patients suffering from a seemingly refractory form of HF. In any case, the improvement of echocardiographic parameters in the CRT group was consistently higher than that observed in the control group during the entire study period.

However, echocardiographic parameters did not improve in a parallel fashion. Mitral regurgitation was reduced at three months and did not materially change subsequently; this is in accordance with studies demonstrating that the reduction of functional mitral regurgitation (MR) is an acute response to the resynchronization of LV wall contraction. In this context, the absence of interaction between aetiology of HF and the effects of CRT on MR is a new finding which indicates that even in IHD patients mitral regurgitation can be significantly reduced by CRT. Changes in LV volumes and EF were maximal at 18 months. Although this study was not designed to verify when the beneficial effects of CRT on reverse remodelling may be considered fully achieved, it is conceivable that a three month window is too short a period of time for the biological effects of CRT (e.g., reverse remodelling) to be completed. We might also infer that we may not expect further LV reverse remodelling in addition to that which has been observed 18 months after implantation of the biventricular device. However, while at 18 months the echocardiographic recording was fixed by the protocol, 29 month is the average value (associated with a relatively large range) for time of end of study evaluations.

The present study demonstrates that the benefits of CRT are not attenuated with time. Such a sustained long term improvement in LV structure and function obtained with CRT strongly supports the hypothesis that dyssynchrony is a fundamental mechanism in the pathogenesis or in the progression of LV dysfunction in patients with systolic HF, broad QRS and left bundle branch block (LBBB).

Effects of CRT in Patients with Ischemic Aetiology

In patients with ischemic heart disease (IHD) undergoing CRT the improvement in LV systolic function and the reduction of LV volumes was significantly attenuated as compared to patients without IHD. This is not an unexpected result, since areas of ischemia or scars within the LV myocardium are likely to be unresponsive to CRT. This finding is also in accordance with the results of the echocardiographic sub-study of the MIRACLE trial. However, no interaction was found between the aetiology of HF and the main clinical outcome of the CARE-HF trial (all-cause mortality or cardiovascular hospital admissions). It is clear that, in addition to reverse remodelling, other beneficial consequences of CRT (such as, for example, a reduction of arrhythmic death) may have contributed to the clinical benefit observed in IHD patients who underwent multi-site pacing.

Effects of CRT in Patients with Advanced Cardiac Dysfunction

Whether CRT may improve myocardial contractility and function in patients with most advanced cardiac dysfunction is a matter of debate. The effects of CRT delivery to patients with a restrictive LV filling pattern and in patients with a reduced TAPSE were also studied; and in fact both parameters are well known echocardiographic predictors of poor prognosis in HF patients. It turned out that the effects of CRT were similar in patients with restrictive LV filling pattern and in patients with a non-restrictive LV filling pattern but that patients with a reduced TAPSE at baseline showed less improvement with CRT than patients with a normal TAPSE. These results can be explained considering that RV dysfunction can characterize a subset of HF patients suffering from global, end-stage, cardiac dysfunction. Such patients might be considered essentially too sick to gain a significant benefit in terms of LV reverse remodelling with CRT.

Long-Term Responsiveness and Inter-Ventricular Dyssynchrony

Refinement of selection criteria for the implantation of biventricular devices can be viewed as a necessary ingredient toward improving patient outcomes and the overall cost-effectiveness of CRT delivery; research is in fact focused on the identification of reliable markers of mechanical dyssynchrony of the LV in the hypothesis that these could be used to predict responsiveness to CRT. At the time the CARE-HF study was designed, information on most of these parameters was still unpublished; therefore the only dyssynchrony parameter included in the echocardiographic protocol of CARE-HF was IVMD. On the other hand, the IVMD has the great advantage of being easily measurable using conventional Doppler echocardiography. Regardless of aetiology of HF, the relationship between IVMD and the probability of a superb response to chronic CRT delivery was essentially linear with a relatively smooth slope (FIG. 5). This means that huge differences in probability of response may not be anticipated on the basis of different IVMD values at baseline. Nonetheless, some useful clinical information may be derived by the analysis of this relationship. In patients with non ischemic etiology the probability of a positive response to CRT at 18 month was never zero, even for negative values of IVMD: low values of IVMD should therefore not be used to negate the benefits of chronic CRT delivery to such patients (e.g. the probability was greater than 50% for IVMD of 20 ms). In the group of IHD patients, the probability of being a long term responder was substantially lower; the risk of non response was therefore high for low values of IVMD (e.g. this risk was nearly 70% for a patient having an IVMD of 40 ms or less).

The following drawings are intended to illustrate some aspects of certain embodiments of the present invention are not to be viewed as limiting as to the full scope of the invention hereof. Further, in certain of the drawings some of the reference numerals are employed to depict different structure, method steps or clinical results and the like. Thus, each drawing should be construed as independent vis-a-vis the reference numerals of other of the drawings.

### BRIEF DESCRIPTION OF THE DRAWINGS

FIG. 1 depicts changes in mitral regurgitation index (mean±95% Cl) in the CRT group of subjects versus the control group of subjects.

FIG. 2 depicts changes in end-diastolic volume (mean+95% Cl) in the CRT group of subjects versus the control group of subjects.

FIG. 3 depicts changes in end-systolic volume (mean+95% Cl) in the CRT group of subjects versus the control group of subjects.

FIG. 4 depicts changes in EF (mean±95% Cl) in the CRT group of subjects versus the control group of subjects.

FIG. 5 depicts the risk of being a CRT non-responder according to treatment, to aetiology of HF (IHD patients vs non IHD patients) and to IVMD at baseline.

FIG. 6 is a diagram depicting an implanted medical device in which the invention may be practiced, in conjunction with a human heart.

FIG. 7 is a block diagram of a multiple-chamber pacing system that implements the invention.

FIG. 8 is a functional schematic diagram of an embodiment of a quadruple chamber implantable pulse generator.

FIG. 9 is a table showing changes in echocardiographic parameters in a control group and in a CRT delivery group (Mean±SD).

FIG. 10 is a table showing the results of a multivariate analysis examining the influence of potential prognostic variables on outcome at 18 months

### DESCRIPTION OF THE ILLUSTRATED EMBODIMENTS

The techniques described below will be presented in the context of CRT delivery to treat HF, contractile disfunction, ischemic heart disease (IHD), reduced ejection fraction (EF), mitral regurgitation, reduced cardiac output (CO), atrial and/or ventricular dysynchrony and other related electrical, perfusion-related, and mechanical performance issues typically experienced by HF patients. Thus, the present is not to be viewed as limited simply to resynchronization of ventricular contractions, and in a broad sense is directed to predicting with a reasonable statistical certainty which HF patients will become reverse remodelling responders (RRR) in response to receipt of CRT. In some embodiments of the invention resynchronization of atrial contractions and/or both atrial and ventricular contractions can be achieved so that at least one portion and/or dimension of the HF patient's heart improves with resulting improvement in the patient's QOL over a longer period of time than might occur otherwise.

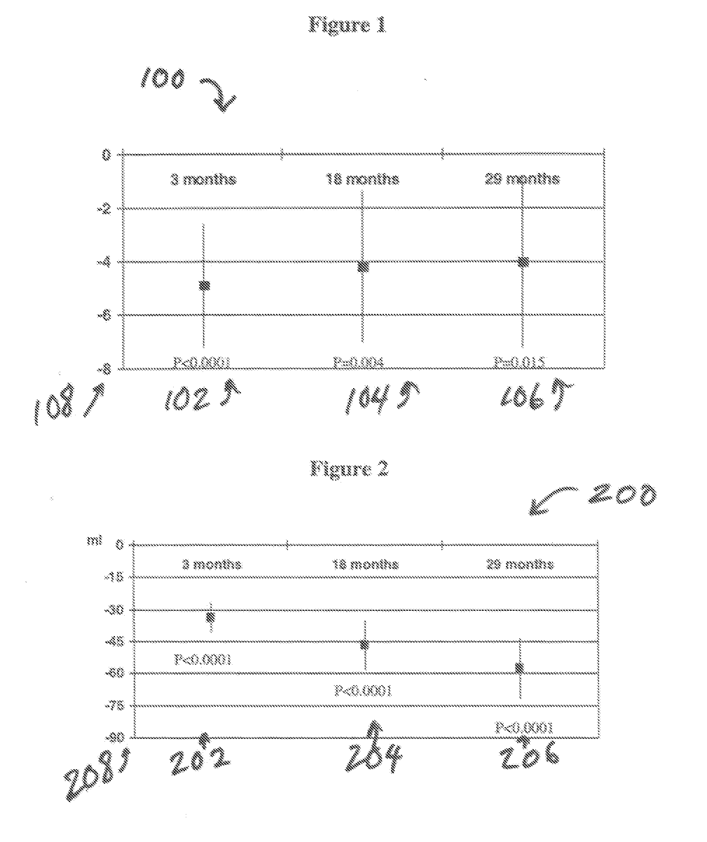

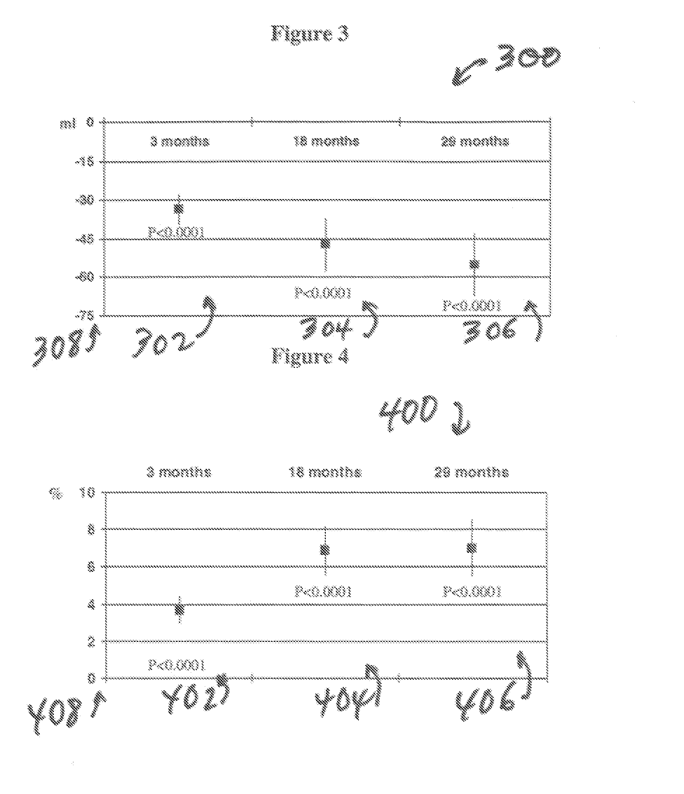

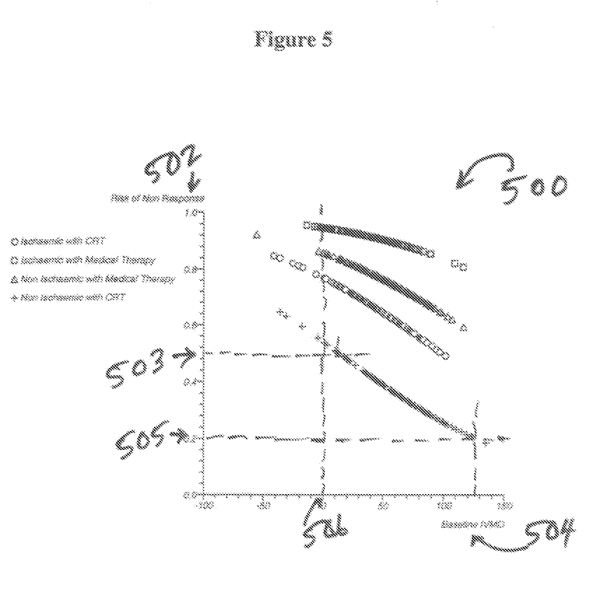

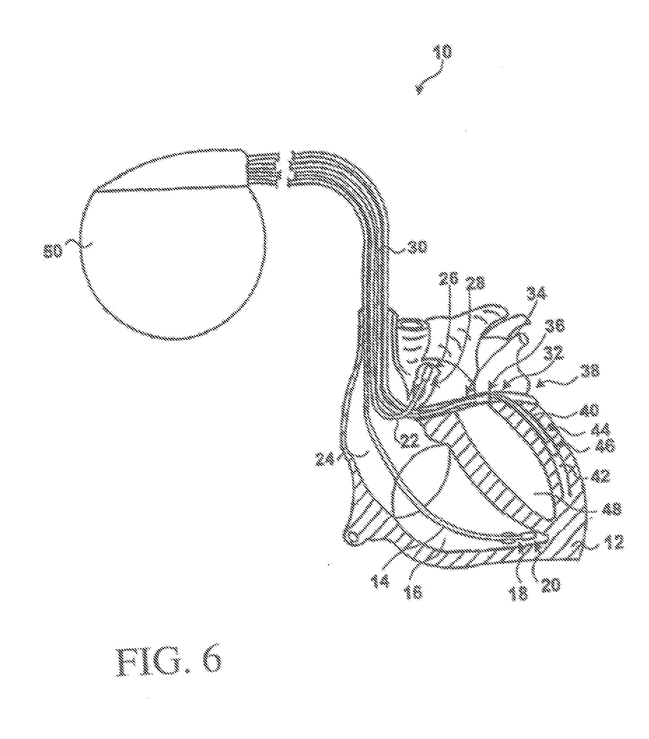

Since FIGS. 1-5 have been discussed in the context of the lengthy summary portion of this patent document the following detailed description will focus nearly exclusively upon FIGS. 6-8 which depict structure for performing CRT in a quadruple chamber implantable pulse generator. Accordingly, referring to FIG. 6, which is a diagram illustrating an implanted medical device 10 for practicing the invention an Implantable Medical Device (IMD) 10, is depicted in conjunction with a human heart 12 and comprises a quadruple-chamber pacing system. RV pacing lead 14 is positioned conventionally in the RV 16 such that its distal end is in the RV apex of heart 12. RV pacing lead 14 carries bipolar electrodes 18,20 that sense electrical signals and can deliver pacing pulses to RV 16. Right atrial (RA) lead 22 is positioned so that its distal end is positioned within the right atrium 24. RA lead 22 carries bipolar electrodes 26,28. Electrodes 26,28 sense electrical activity in right atrium 24 and may also deliver pacing pulses to right atrium 24. Left atrial (LA) lead 30 is passed through right atrium 24 so that the distal end of lead 30 is positioned in the coronary sinus 32. Electrodes 34,36 on LA lead 30 sense electrical activity in the left atrium 38 and may also deliver pacing pulses to left atrium 38. LV lead 40 is positioned via coronary sinus 32 in a cardiac vein 42, such as the middle or great cardiac vein. Distal electrodes 44,46 on LV lead 40 are positioned for pacing and sensing with respect to the LV 48. Leads 14,22,30,40 connected to pacemaker 50 in a conventional manner. Pacemaker 50 receives electrical signals sensed by electrodes in the atria and ventricles, and is adapted to deliver pacing pulses to the atria (RA,LA) and/or ventricles (RV,LV). In particular, pacemaker 50 can receive an atrial sense from electrodes 26,28, and following a predetermined AV delay, delivers one or more bi-ventricular pace stimulus. Pacemaker 50 delivers a bi-ventricular pace by pacing RV 16 and LV 48 to cause cardiac resynchronization. The ventricles may be paced simultaneously, or one ventricle may be paced before the other. As will be described in more detail below, pacemaker 50 does not deliver bi-ventricular pacing after every atrial sense. Rather, pacemaker 50 adjusts cardiac resynchronization to acclimate the patient to the therapy.

Implanted medical device 10 merely serves as an exemplary device that may use the techniques of the invention. However, the invention is not limited to the device shown. For example, the invention may be practiced with unipolar electrodes rather than bipolar electrodes. The invention may further be practiced in a less complicated device, such as a device with two ventricular leads with sensing/pacing electrodes and a single atrial lead with a sensing electrode. Conversely, the invention may be practiced in a more complicated device as well, such as a device with each of the leads having more electrodes than are shown in FIG. 6.

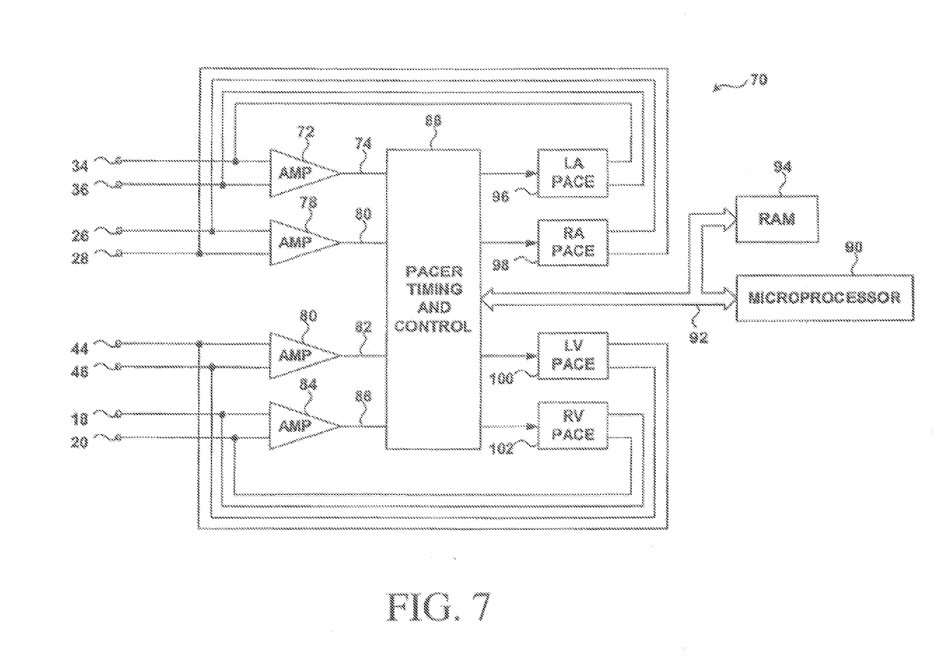

FIG. 7 is a block diagram of a system 70 that implements the invention. FIG. 7 is exemplary of the type of device in which the invention may be practiced, but the invention may be practiced in a wide variety of device implementations. Electrodes 34,36 are located proximal to left atrium 38 and are coupled to a P-wave amplifier 72 in pacemaker 50. P-wave amplifier 72 can take the form of an automatic gain controlled amplifier (AGC) providing an adjustable sensing threshold as a function of the amplitude of the P-wave sensed by electrodes 34,36. Amplifier 72 generates a signal on P-out line 74 whenever the signal sensed between electrodes 34,36 exceeds the sensing threshold. In like fashion, electrodes 26,28 are located proximal to right atrium 24 and are coupled to a P-wave amplifier 76. Amplifier 76 generates a signal on P-out line 78 whenever the signal sensed between electrodes 26,28 exceeds the sensing threshold. Similarly, electrodes 44,46, located proximal to LV 48, are coupled to an R-wave amplifier 80, and electrodes 18,20, located proximal to RV 16, are coupled to another R-wave amplifier 84. Amplifier 80 generates a signal on R-out line 82 whenever the signal sensed between electrodes 44,46 exceeds the sensing threshold, and amplifier 84 generates a signal on R-out line 86 whenever the signal sensed between electrodes 18,20 exceeds the sensing threshold. Pacer timing and control circuitry 88 receives the signals from P-out lines 74,78 and R-out lines 82,86. Pacer timing and control circuitry 88 can include programmable digital counters that control the basic time intervals associated with modes of single-chamber pacing and multiple-chamber pacing. Microprocessor 90 regulates pacer timing and control circuitry 88 by, for example, determining the appropriate pacing therapy and determining the amplitude of the cardiac pacing pulses. Microprocessor 90 loads pacing instructions to pacer timing and control circuitry 88 via bus 92. As will be described in more detail below, the instructions may include parameters pertaining to CRT timing parameters (e.g., A-V intervals, V-V intervals, etc.). These parameters may be programmed by the patient's physician and stored in memory such as random access memory (RAM) 94. The physician may, for example, program the parameters with a programmer, which communicates with implanted medical device 10 via telemetry. When cardiac resynchronization or other pacing is indicated, pacer timing and control circuitry 88 triggers one or more pace pulse generators 96,98,100,102. Pace pulses are transmitted from pace pulse generators 96,98,100,102 to cardiac tissue via the corresponding electrodes. For example, a pacing pulse generated by RV pace pulse generator 102 is delivered to RV 16 via electrodes 18 and 20. In a bi-ventricular pace, pacer timing and control circuitry 88 triggers pacing pulses delivered from pace pulse generators 100 and 102. The invention is not to be deemed limited to the system 70 depicted in FIG. 7. For example, the invention may be practiced using a chronically implanted pulse generator that also provides defibrillation or cardioversion therapies. In addition, the delivery of CRT can be applied using a partially implanted pacing system in an acute or temporary manner. The invention may also be practiced, for example, in a system that provides for atrial sensing but not for atrial pacing, or in a system that includes no electrodes to sense or pace left atrium 38. Moreover, microprocessor 90 and pacer timing and control circuitry 88 are depicted in FIG. 7 as logically distinct components, but the invention is not limited to such an arrangement. The invention can be implemented in a pacemaker that combines the functions of microprocessor 90 and pacer timing and control circuitry 88 in a single component. In particular, in some embodiments, the functions of pacer timing and control circuitry 88 may be programmed features of microprocessor 90. In the following described exemplary embodiments, determinations concerning cardiac resynchronization will be made by microprocessor 90, but such determinations may be made by another component such as pacer timing and control circuitry 88 or another processor not shown in FIG. 7. Microprocessor 90 may be programmed to provide cardiac resynchronization in some situations but not in others. For example, microprocessor 90 may providing cardiac resynchronization to a heart in response to one sensed event, but may refrain from delivering CRT in response to another sensed event (e.g., a blood oxygen sensor, an accelerometer coupled to a portion of contractile tissue, a pressure sensor disposed in fluid communication with one or more chambers of the heart, etc.).

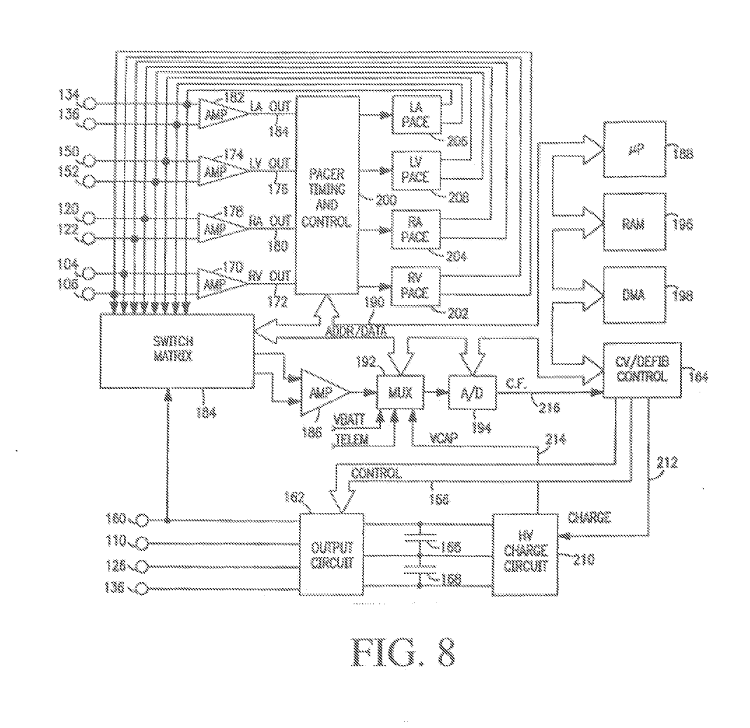

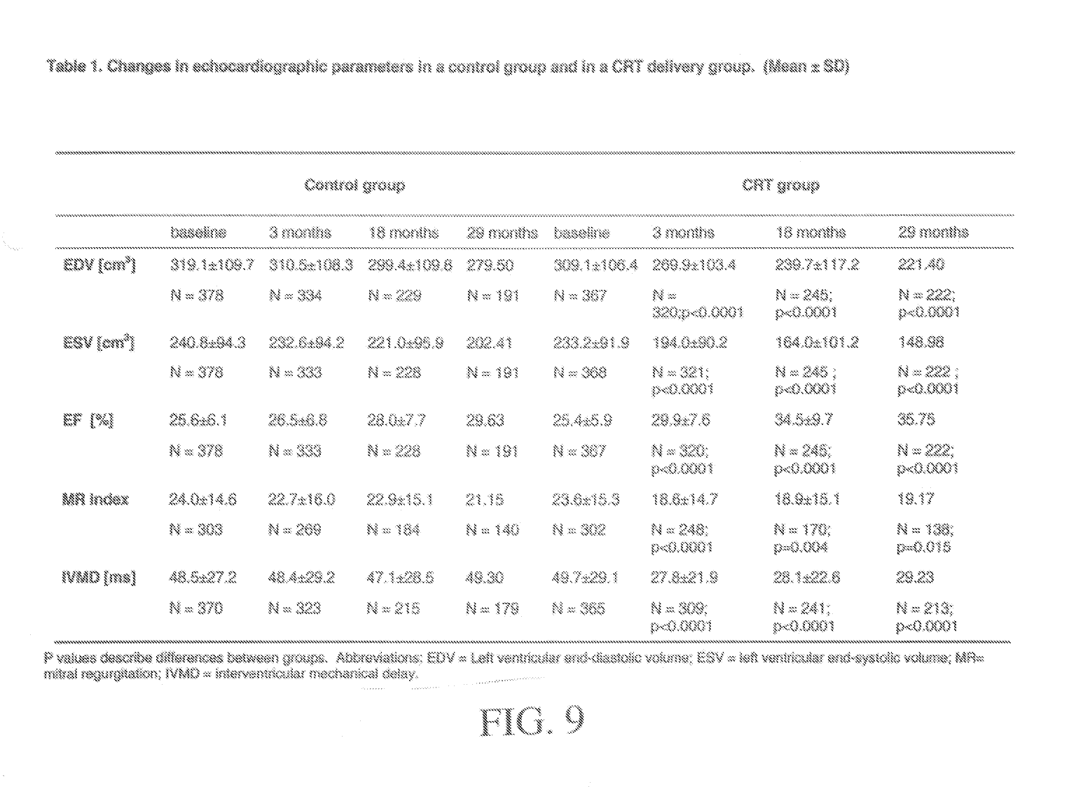

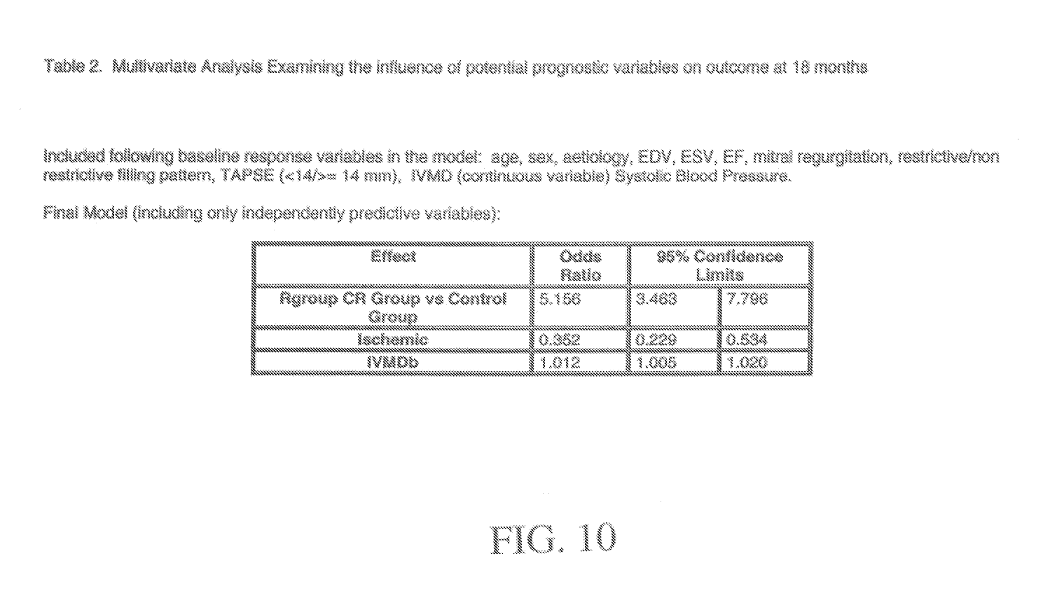

FIG. 8 illustrates, in the form of a functional schematic diagram, an embodiment of an IMD 10 capable of delivering CRT pursuant to certain aspects of the present invention. This diagram should be taken as exemplary of the type of device in which various embodiments of the present invention may be embodied, and not as limiting, as it is believed that the invention may be practiced in a wide variety of device implementations, including cardioverter and defibrillators which do not provide anti-tachycardia pacing therapies. The IMD 10 is provided with an electrode system. Electrode 160 in FIG. 8 can include an uninsulated portion of the housing 18 of IMD 10. Electrodes 110,126,136,160 can, as noted previously be coupled to high voltage output circuit 162, which optionally includes a pair of high voltage switches controlled by cardioversion/defibrillation (CV/defib) control logic 164 via control bus 166. Switches disposed within circuit 162 determine which electrodes are employed and which electrodes are coupled to the positive and negative terminals of a capacitor bank (which includes capacitors 166,168) during delivery of defibrillation pulses. Electrodes 104,106 are located on or in the RV of the patient and are coupled to the R-wave amplifier 170, which can take the form of an automatic gain controlled (AGC) amplifier, or a digital equivalent thereof, providing an adjustable sensing threshold as a function of the measured R-wave amplitude. A signal is generated on R-out line 172 whenever the signal sensed between electrodes 104 and 106 exceeds the present sensing threshold. Similarly, electrodes 150,152 are located in electrical communication with the LV of the patient coupled to the R-wave amplifier 174, which can take the form of an AGC (or digital) amplifier providing an adjustable sensing threshold as a function of the measured R-wave amplitude. A signal is generated on R-out line 176 whenever the signal sensed between electrodes 150,152 exceeds the present sensing threshold. Electrodes 120,122 couple the right atrium of the patient to P-wave amplifier 178, which again can take the form of an AGC amplifier providing an adjustable sensing threshold as a function of the measured P-wave amplitude. A signal is generated on P-out line 180 whenever the signal sensed between electrodes 120,122 exceeds the present sensing threshold. Similarly, electrodes 134,136 couple the left atrium of the patient to P-wave amplifier 182 of the types previously noted and a signal is generated on P-out line 184 whenever the signal sensed between electrodes 134,136 exceeds the present sensing threshold. The general operation of R-wave and P-wave amplifiers 170,174,178,182 can correspond to that disclosed in U.S. Pat. No. 5,117,824 to Keimel et al., hereby incorporated by reference herein in its entirety. Switch matrix 184 is used to select which of the available electrodes are coupled to wide band (0.5-200 Hz) amplifier 186 for use in digital signal analysis. Selection of electrodes is controlled by microprocessor 188 via data/address bus 190, which selections may be varied as desired. Signals from the electrodes selected for coupling to bandpass amplifier 186 are provided to multiplexer 192, and thereafter converted to multi-bit digital signals by A/D converter 194, for storage in random access memory 196 under control of direct memory access circuit 198. Microprocessor 188 may employ digital signal analysis techniques to characterize the digitized signals stored in random access memory 196 to recognize and classify the patient's heart rhythm employing any of the numerous signal processing methodologies known to the art. The remainder of the circuitry is dedicated to the provision of cardiac pacing, cardioversion and defibrillation therapies, and, for purposes of the present invention can correspond to circuitry known to those skilled in the art.

The following exemplary apparatus is disclosed for accomplishing pacing, cardioversion and defibrillation functions. Pacer timing/control circuitry 200 can include programmable digital counters which control the basic time intervals associated with DDD, VVI, DVI, VDD, AAI, DDI (as well as rate-responsive modes of the foregoing) and other modes of single and multi-chamber pacing well known to the art. Circuitry 200 can also control escape intervals associated with anti-tachyarrhythmia pacing in both the atrium and the ventricle, employing any anti-tachyarrhythmia pacing therapies known to the art.

Intervals defined by pacing circuitry 200 include atrial and ventricular pacing escape intervals, the refractory periods during which sensed P-waves and R-waves are ineffective to restart timing of the escape intervals and the pulse widths of the pacing pulses. The durations of these intervals are determined by microprocessor 188, in response to stored data in memory 196 and are communicated to pacing circuitry 200 via address/data bus 190. Pacer circuitry 200 also determines the amplitude of the cardiac pacing pulses under control of microprocessor 188.

During pacing, escape interval counters within pacer timing/control circuitry 200 are reset upon sensing of R-waves and P-waves as indicated by a signals on lines 172,176,180,184 and in accordance with the selected mode of pacing on time-out trigger generation of pacing pulses by pacer output circuitry 202,204,206,208, which are coupled to electrodes 104,106,120,122,134,136,150,152. Escape interval counters are also reset on generation of pacing pulses and thereby control the basic timing of cardiac pacing functions, including anti-tachyarrhythmia pacing. The durations of the intervals defined by escape interval timers are determined by microprocessor 188 via data/address bus 190. The value of the count present in the escape interval counters when reset by sensed R-waves and P-waves may be used to measure the durations of R-R intervals, P-P intervals, P-R intervals and R-P intervals, which measurements are stored in memory 196 and used to detect the presence of tachyarrhythmias.

IMD 10 can provide bi-ventricular pacing or bi-atrial pacing and/or both and optionally can include offset timing between complementary chambers of the heart. Further, IMD 10 may provide bi-ventricular pacing or bi-atrial pacing in combination with other pacing. For example, IMD 10 may pace one atrium and both ventricles, or one ventricle and both atria. In bi-atrial pacing, IMD 10 delivers pacing pulses to the atria, the pulses separated by a delay sometimes referred to as an A1-A2 interval. In bi-ventricular pacing, IMD 10 may deliver pacing pulses to the ventricles separated by a similar interval, sometimes referred to as a V1-V2 interval. Pacer timing/control circuitry 200 controls the durations of the A1-A2 interval and the V1-V2 interval, as applicable, as well as variations thereof (e.g., a single atrial event, A1, to trigger a left or RA paced event and a pair of ventricular intervals, V1-V2). Microprocessor 188 typically operates as an interrupt driven device, and is responsive to interrupts from pacer timing/control circuitry 200 corresponding to the occurrence of sensed P-waves and R-waves and corresponding to the generation of cardiac pacing pulses. Those interrupts are provided via data/address bus 190. Any necessary mathematical calculations to be performed by microprocessor 188 and any updating of the values or intervals controlled by pacer timing/control circuitry 200 take place following such interrupts.

Detection of atrial or ventricular tachyarrhythmias, as employed in the present invention, may correspond to tachyarrhythmia detection algorithms known in the art. For example, the presence of an atrial or ventricular tachyarrhythmia may be confirmed by detecting a sustained series of short R-R or P-P intervals of an average rate indicative of tachyarrhythmia or an unbroken series of short R-R or P-P intervals. The rate of onset of the detected high rates, the stability of the high rates, and a number of other factors known in the art may also be measured at this time. Appropriate ventricular tachyarrhythmia detection methodologies measuring such factors are described in U.S. Pat. No. 4,726,380 issued to Vollmann, U.S. Pat. No. 4,880,005 issued to Pless et al., and U.S. Pat. No. 4,830,006 issued to Haluska et al., all incorporated by reference herein, each in its respective entirety.

In the event an atrial or ventricular tachyarrhythmia is detected and an anti-tachyarrhythmia pacing regimen is desired, appropriate timing intervals for controlling generation of anti-tachyarrhythmia pacing therapies are loaded from microprocessor 188 into the pacer timing and control circuitry 200, to control the operation of the escape interval counters therein and to define refractory periods during which detection of R-waves and P-waves is ineffective to restart the escape interval counters. Alternatively, circuitry for controlling the timing and generation of anti-tachycardia pacing pulses as described in U.S. Pat. No. 4,577,633, issued to Berkovits et al., U.S. Pat. No. 4,880,005, issued to Pless et al., U.S. Pat. No. 4,726,380, issued to Vollmann et al., and U.S. Pat. No. 4,587,970, issued to Holley et al., all of which are incorporated herein by reference in their entireties, may also be employed.

In the event that generation of a cardioversion or defibrillation pulse is required, microprocessor 188 may employ an escape interval counter to control timing of such cardioversion and defibrillation pulses, as well as associated refractory periods. In response to the detection of atrial or ventricular fibrillation or tachyarrhythmia requiring a cardioversion pulse, microprocessor 188 activates cardioversion/defibrillation control circuitry 164, which initiates charging of high voltage capacitors 166,168 via charging circuit 210, under the control of high voltage charging control line 212. The voltage on the high voltage capacitors is monitored via VCAP line 214, which is passed through multiplexer 192 and in response to reaching a predetermined value set by microprocessor 188, results in generation of a logic signal on Cap Full (CF) line 216 to terminate charging. Thereafter, timing of the delivery of the defibrillation or cardioversion pulse is controlled by pacer timing/control circuitry 200. Following delivery of the fibrillation or tachycardia therapy microprocessor 188 returns the device to cardiac pacing mode and awaits the next successive interrupt due to pacing or the occurrence of a sensed atrial or ventricular depolarization. Several embodiments of appropriate systems for the delivery and synchronization of ventricular cardioversion and defibrillation pulses and for controlling the timing functions related to them are disclosed in U.S. Pat. No. 5,188,105 to Keimel, U.S. Pat. No. 5,269,298 to Adams et al., and U.S. Pat. No. 4,316,472 to Mirowski et al., hereby incorporated by reference herein, each in its respective entirety. Any known cardioversion or defibrillation pulse control circuitry is believed to be usable in conjunction with various embodiments of the present invention, however. For example, circuitry controlling the timing and generation of cardioversion and defibrillation pulses such as that disclosed in U.S. Pat. No. 4,384,585 to Zipes, U.S. Pat. No. 4,949,719 to Pless et al., or U.S. Pat. No. 4,375,817 to Engle et al., all hereby incorporated by reference herein in their entireties, may also be employed.

Continuing to refer to FIG. 8, delivery of cardioversion or defibrillation pulses is accomplished by output circuit 162 under the control of control circuitry 164 via control bus 166. Output circuit 162 determines whether a monophasic or biphasic pulse is delivered, the polarity of the electrodes and which electrodes are involved in delivery of the pulse. Output circuit 162 also includes high voltage switches which control whether electrodes are coupled together during delivery of the pulse. Alternatively, electrodes intended to be coupled together during the pulse may simply be permanently coupled to one another, either exterior to or interior of the device housing, and polarity may similarly be pre-set, as in many currently available implantable defibrillators. An example of output circuitry for delivery of biphasic pulse regimens to multiple electrode systems may be found in the above-cited patent issued to Mehra and in U.S. Pat. No. 4,727,877 to Kallok, hereby incorporated by reference herein in its entirety.

An example of circuitry which may be used to control delivery of monophasic pulses is disclosed in U.S. Pat. No. 5,163,427 to Keimel, also incorporated by reference herein in its entirety. Output control circuitry similar to that disclosed in U.S. Pat. No. 4,953,551 to Mehra et al. or U.S. Pat. No. 4,800,883 to Winstrom, both incorporated by reference herein in their entireties, may also be used in conjunction with various embodiments of the present invention to deliver biphasic pulses.

Although FIG. 8 depicts one electrode per cardiac chamber, the invention is not limited to a single pacing electrode per chamber. Rather, the invention may be applied to multi-chamber pacing in which two or more electrodes per chamber. For example, the invention may be applied to a bi-ventricular pacing system that includes a single electrode in the RV, but three electrodes placed around the LV, such as the LV anterior-septum wall, the LV lateral free wall, and the LV posterior free wall. Multiple-site electrode placement with respect to a single cardiac chamber may, for some patients, result in more homogenous activation and homogenous mechanical response.

While this patent document describes, teaches, illustrates and claims a discrete few embodiments and forms of the invention, those of skill in the art will readily recognize that insubstantial changes can be made with respect to the invention without departing from the spirit and scope of the invention. All such related embodiments are deemed expressly covered by the instant disclosure only as limited by the appended claims.

1. A method of predicting whether a subject can reasonably be expected to experience beneficial cardiac reverse remodelling due to delivery of a cardiac resynchronization therapy (CRT), comprising:
measuring an inter-ventricular mechanical delay (IVMD) of a heart failure patient during an episode of normal sinus rhythm (NSR); and
based at least in part upon the magnitude of the measured IVMD, predicting whether the patient can be expected to successfully experience beneficial reverse remodelling of at least a part of the patient's heart in response CRT delivery.

2. A method according to claim 1, wherein the IVMD is measured with an echocardiographic measurement apparatus.

3. A method according to claim 1, wherein in lieu of the episode of NSR the IVMD is measured during at least one paced activation of at least one of the ventricles of the HF patient.

4. A method according to claim 1, further comprising delivering a CRT to the HF patient.

5. A method according to claim 4, wherein the CRT is delivered to the HF patient via one of a programmable implantable pulse generator and an implantable cardioverter-defibrillator (ICD).

6. A method according to claim 4, the CRT is delivered to the HF patient on at least a substantially chronic basis.

7. A method according to claim 1, wherein the HF patient has been diagnosed as suffering from myocardial ischemia and the magnitude of the measured IVMD has a magnitude of about 100 milliseconds and wherein the prediction is approximately fifty percent positive (50%) and fifty percent (50%) negative.

8. A method according to claim 1, wherein the HF patient has been diagnosed as suffering from myocardial ischemia and the magnitude of the measured IVMD has a magnitude of about zero milliseconds and wherein the prediction is approximately twenty percent positive (20%) and eighty percent (80%) negative 9. A method according to claim 1, wherein the HF patient has been diagnosed as not previously suffering from myocardial ischemia and the magnitude of the measured IVMD is between about zero milliseconds and about one hundred twenty-five (125) milliseconds and wherein the result of the prediction is approximately fifty percent (50%) positive and fifty percent (50%) negative at approximately the zero millisecond magnitude and increases substantially linearly to about approximately eighty percent (80%) positive and twenty percent (20%) negative at approximately the one hundred twenty-five (125) millisecond magnitude.

9. A method according to claim 1, wherein the portion of the heart comprises a left ventricular chamber.

10. A method according to claim 1, wherein the reverse remodelling includes at least one of a relatively increased cardiac output (CO) metric, a relatively increased ejection fraction (EF) metric, a relatively reduced end-systolic volume (ESV), a relatively reduced end-diastolic volume (EDV) metric, a relatively decreased QRS duration metric, a relatively reduced incidence of mitral regurgitation.

11. A method according to claim 1, wherein the HF patient is indicated as one of a New York Heart Association (NYHA) Class III and NYHA Class IV patient.

12. An apparatus useful in predicting whether a heart failure (HF) patient can reasonably be expected to experience beneficial cardiac reverse remodelling due to delivery of a cardiac resynchronization therapy (CRT), comprising:
means for measuring an inter-ventricular mechanical delay (IVMD) of a HF patient during an episode of normal sinus rhythm (NSR); and
means for predicting whether the HF patient can be expected to successfully experience reverse remodelling of at least a part of the HF patient's heart in response to delivery of a CRT based at least in part upon the magnitude of the measured IVMD.

13. An apparatus according to claim 12, wherein the IVMD is measured with an echocardiographic measurement apparatus.

14. An apparatus according to claim 12, wherein in lieu of the episode of NSR the IVMD is measured during at least one paced activation of at least one of the ventricles of the HF patient.

15. An apparatus according to claim 12, further comprising means for delivering a CRT to the HF patient.

16. An apparatus according to claim 15, wherein the CRT is delivered to the HF patient via one of a programmable implantable pulse generator and an implantable cardioverter-defibrillator (ICD).

17. An apparatus according to claim 12, wherein the CRT is delivered to the patient on at least a substantially chronic basis.

18. An apparatus according to claim 12, wherein the HF patient is indicated as one of a New York Heart Association (NYHA) Class III and NYHA Class IV patient.

19. An apparatus according to claim 12, further comprising means for indicating whether the HF patient is more or less likely to experience any beneficial reverse remodelling result from delivery of a CRT.

20. An apparatus according to claim 19, wherein the means for indicating includes at least one of: an radio frequency (RF) transmitted signalling apparatus, a wired-signalling apparatus, a haptic signalling apparatus, an illuminated display apparatus, a graphical display apparatus, a percentage-based display apparatus, a bar graph-type display, a numerical display, a textural display.
